(19) United States
(12) Patent Application Publication
(10) Pub. No.: US 2012/0310310 A1
(43) Pub. Date: Dec. 6, 2012

(54) Materials and accessories for the application of antibacterial photodynamic therapy

(76) Inventor: Peter PATEROK, Monchengladbach (DE)

(21) Appl. No.: 13/557,319

(22) Filed: Jul. 25, 2012

Related U.S. Application Data
(63) Continuation of application No. PCT/DE2011/000073, filed on Jan. 26, 2011.

(30) Foreign Application Priority Data
Jan. 27, 2010 (DE) 10 2010 006 035.6

(51) Int. Cl. A61N 5/06

(52) U.S. Cl. 607/93

(57) ABSTRACT

An apparatus for carrying out antibacterial photodynamic therapy that includes an illumination means and a means for dispensing the photosensitizer, wherein the means for dispensing the photosensitizer is adapted in such a manner that the photosensitizer is dispensed in spatial proximity to the body region to be treated.

### CROSS-REFERENCE TO RELATED APPLICATIONS

This utility patent application filed under 35 USC 111 is a continuation of pending international application PCT/DE2011/000073 filed on Jan. 26, 2011 and claims priority to German patent application 102010006035.6 filed on Jan. 27, 2010, which are both incorporated by reference herein.

### TECHNICAL FIELD

The present invention relates to materials and accessories for the application of photodynamic therapy.

### BACKGROUND

The principle of photodynamic therapy (PDT) is based on selective photobiostimulation; in this way, light from a light source can be absorbed by a suitable chromophore. Herein, the chromophore absorbs the photon energy of the light beam. In the same way, irradiation of a photosensitizer with a wavelength corresponding to the absorption maximum results in the dye absorbing the photon energy. Herein, the photosensitizer assumes the singlet state. However, from the excited state it can revert to the fundamental state while energy is emitted in the form of fluorescence. Or else it can convert into a triplet state, in which it can give off energy to another triplet molecule.

Such a molecule in the fundamental state is oxygen. The energy is now transferred from the triplet state of the dye to the oxygen, which is excited, and the oxygen is elevated into the highly toxic singlet oxygen state. Such a singlet oxygen is highly toxic and can massively damage the cell core. However, it is also short lived and cannot diffuse very far in this period of time. This is why the cellular damage is limited to the area in which the photosensitizer is enriched and the light is irradiated. Currently used photosensitizers absorb light in wavelengths between 630 nm and 690 nm., e.g., the absorption maximum of methylene blue is at 664 nm. There are also other photosensitizers, some of them effective in other wavelength ranges, such as aminolevulinic acid, indocyanine green, phthalocyanine, photolone, a chlorine derivative, erythrosines, focsan and hypericine. This list is not exhaustive. The marker germs were most substantially reduced in our experience when the wavelengths were optimally adapted.

Also, irradiation alone does not result in a reaction, nor does dying alone lead to a reaction. The crucial factor here is the cooperation of dying and irradiation. To achieve an optimal dose, the light applicators are necessary to achieve spatially homogeneous irradiation of the tissue surface, and reliable light dosimetry is also necessary.

Light sources or light applicators can be of various types depending on each respective application area. It must only be insured that light of the relevant wavelength impinges on surfaces marked with the photosensitizer.

Generally, antibacterial therapy can be carried out with PDT as follows. What is needed is a photosensitizer, a light source having its wavelength and power adapted to the photosensitizer, and oxygen in molecular form. After dying the microorganisms, such as bacteria, the excess dye is flushed away, and the photosensitizer is then exposed to light and activated, whereby singlet oxygen is formed, which kills off the microorganisms.

The antibacterial photodynamic therapy is already known in applied dentistry, namely with root treatment and the treatment of periodontal pockets, i.e., in periodontology. The light source used herein is a so-called optical fiber, which can be introduced into the periodontal pockets. Such a light source, however, cannot be used in all cases for the treatment of other body cavities contaminated by bacteria. The use of optical fibers is sometimes not effective, since the surfaces irradiated are too small.

Furthermore, simple flushing to remove the excess dye in some body cavities leads to the bacterial infection being transported to further, hidden, body cavities. This extremely deleterious effect should be avoided at all cost. Thus simple flushing of the oral, pharyngeal and nasal cavities simply leads to the bacteria being generally spread and thus also carried to areas which were previously free of bacteria. Furthermore, certain areas of the body cannot be treated with the aid of photodynamic therapy at all.

### SUMMARY

It is thus the object of the present invention to provide an apparatus and a method providing photodynamic therapy, or improved photodynamic therapy, in the nasal, oral and pharyngeal cavities and other regions of the body, which have mucous membranes or young cicatricial tissue, or to improve such photodynamic therapy, wherein the distribution of pathogens is to be prevented.

The object is achieved according to the present invention by an apparatus for carrying out antibacterial photodynamic therapy, characterized in that the apparatus comprises an illumination means and a means for dispensing the photosensitizer, wherein the illumination means is an optical fiber irradiating the light in the desired frequency through its side walls, and the means for dispensing the photosensitizer is configured in such a manner that the photosensitizer is dispensed in spatial proximity to the body region to be treated, and wherein a flushing device for removing the excess dye, i.e., the photosensitizer, comprises a suction device for largely sucking in the flushing agent and the excess dye dissolved therein, and the means for dispensing the photosensitizer, preferably a tube, sheaths the light irradiating portion of the illumination means, wherein the sheathing material at least partially transmits the light irradiated from the illumination means.

This apparatus enables almost optimal performance of photodynamic therapy since the photosensitizer is dispensed in spatial proximity to the body regions to be treated. Cells, i.e. bacteria and microorganism cells, are quickly dyed, which are then to be killed off by irradiation. The photosensitizer is thus not “wasted” or introduced to locations in the body in which it should not become effective.

In another embodiment of the apparatus according to the present invention, the illumination means at least partially comprises an optical fiber, which irradiates the light of the desired frequency to the body region to be treated. Such optical fibers are light emitting apparatus comprising a light source remote from the area to be illuminated with it. This means that the light source generates the light which is then emitted in a different location, normally far removed.

Usually, the optical fibers comprise a core in the central area and a sheath with a predefined refractive index, which is lower, for example, than that of the core, on the periphery of the core. Optical fibers are known which transport light to the end of the fiber. Other optical fibers radiate the light from the side walls and thus light the entire distance over which the optical fiber extends.

Optical fibers are disclosed in JP-A-6-118244, which emit light from the entire lateral surface or only from a certain, i.e. limited surface area. These optical fibers are equipped with a core and a sheath, both of transparent materials, wherein the core comprises a polymer primarily consisting of a polymethyl acrylate. The sheath is of two different portions, wherein one consists of a copolymer on the basis of vinylidene fluoride, comprising 50 to 90 mol % vinylidene fluoride and 10 to 50 mol % tetrafluoroethylene. The other portion comprises at least one of the other polymers having a refractive index higher than that of the copolymer on the basis of vinylidene fluoride, preferably a polymer that is the same or similar to the polymer forming the core.

However, there are many other materials that have the desired properties of optical fibers. For example, JP-A-10-142428 discloses a light illumination bar and JP-B-4-70604 discloses optical fibers of flexible elements.

In a further particularly preferred embodiment of the invention, a tube or a tube-like structure is the means to dispense the photosensitizer, which conducts the photosensitizer from a source via the tube to the body regions to be treated. Wherein the tube can have at least one outlet to dispense the photosensitizer, but can also have an indefinite maximum number of outlets to dispense the photosensitizer.

This tube or this tube-like structure contains the photosensitizer and guides it to the predetermined body regions. The tube or the tube-like structure can have a plurality of outlets for the photosensitizer, through which the latter can then be dispensed to the surrounding tissue. This embodiment of the invention ensures precise dispensing and dosing of the photosensitizer to the predetermined body regions.

In a further embodiment of the present invention, the means for dispensing the photosensitizer, preferably a tube, sheaths the light emitting portion of the illumination means, wherein the sheathing material at least partially transmits the light emitted by the illumination means. Thus, a further sheathing of the illumination means is provided. If the illumination means is an optical fiber, a further sheathing is thus provided, carrying the photosensitizer within it, around the core and the sheathing of the core of the above mentioned transparent material. It must, however, be ensured that this sheathing does not change the optical properties of the optical fiber in such a way that the emitted light is irradiated in a different wavelength range and thus compromises the effectiveness of the photodynamic therapy.

In a further embodiment of the present invention, the illumination means, preferably an optical fiber, is coated with the photosensitizer, which is then slowly released from the former and adheres to the microorganisms and bacteria.

In a completely different embodiment of the present invention, the present invention is characterized in that the sheathing material is of a fabric which has absorbed the photosensitizer and then in turn dispenses it to the body regions to be treated. A fabric web can be envisaged, which sheaths an optical fiber. This fabric is immersed in the photosensitizer in preparation, wherein it is impregnated with the latter. In the application in the context of photodynamic therapy, the photosensitizer is slowly dispensed to the surrounding tissue, where it spreads. The direction of spreading within the surrounding tissue is effected by any fluid flow present there and by diffusion forces.

In a preferred embodiment of the invention, the apparatus according to the present invention (applicator) additionally comprises a flushing device for removing excess dye. Such an apparatus (applicator), i.e., an embodiment for carrying out the antibacterial photodynamic therapy, specially adapted to the body portions to be treated, enhances the effectiveness of the treatment thanks to the combination of the flushing device and the light source. The spatial proximity of the flushing device and the light source ensures precision-guided treatment at the desired location.

Also, the temporal sequence of flushing and exposure to light can be precisely tuned with respect to each other. It should also be noted, that when water is used for flushing, it can also serve as a light guide. This so-called Tyndall effect should be used for treating locations that are difficult to access.

In a particularly preferred embodiment, the apparatus (applicator) according to the present invention, for carrying out antibacterial photodynamic therapy, comprises the flushing device and a suction device, which sucks most of the flushing fluid back in. The excess dye must be removed with great care, since it can actually hinder the exposure to light and activation if excessive layers of it are present on the tissue. For optimum treatment, the dye should be absorbed by the tissue in the cell wall. Any dye not absorbed by the cell wall should be removed. As repeatedly mentioned, this is done by simple flushing. Water is suitable as a flushing fluid. Too much flushing can flush the bacteria into body cavities in which they were not present before and can thus even lead to spreading of the bacterial infection. If directly after flushing, the flushing liquid, such as water, is sucked back in, it cannot disperse. If the flushing and suction devices are in close proximity, the excess dye is also sucked in.

In a particularly preferred embodiment of the apparatus (applicator) of the present invention, the latter comprises an ultrasonic nebulizer for applying the flushing fluid to the desired locations in an extremely finely distributed form. Hereby the amount of flushing fluid introduced can be better dosed and reduced, while the effect is the same. Ultrasonic nebulizers are commercially available in the desired size and can easily be incorporated in the apparatus (the applicator). The finely distributed flushing fluid particles additionally ensure that light is scattered during exposure to light and thus the dyed cell walls are more effectively irradiated.

In a further embodiment, the apparatus (applicator) has a y-shaped configuration so that it is suitable for use in the nasal cavity. Each of the two ends of the y-shaped applicator (the apparatus) has its own flushing device and its own light source. The application (the apparatus) will be introduced into the nose in such a way that one end is introduced into each nostril. It is thus ensured that the entire nasal mucous membrane is treated simultaneously.

In a particularly preferred embodiment of the present invention, it is characterized in that the flushing and suction devices of one applicator end (the end of the apparatus) flushes whenever the flushing and suction devices of the other applicator end suck in the flushing fluid. A direction of flow of the flushing fluid from one applicator end to the other applicator end is thus created and distribution of the flushing fluid contaminated with bacteria in the entire nasal cavity, the sinuses and the other apertures present there is avoided. By changing the direction of flow, the flushing operation becomes even more effective.

In a further embodiment of the present invention, the flushing device is configured as an inhalation apparatus. By these means, flushing fluid is sucked in by inhalation and the excess dye is then flushed away.

In another embodiment of the present invention, the apparatus (applicator) has a shape adapted to the dental ridge and is thus optimized for application in the pharyngeal region. Also in this embodiment, the flushing and suction devices can be present at each applicator end (apparatus end) and define a direction of flow of the flushing fluid as they are alternately operated. This embodiment can be configured as an occlusion rim and can also have a spatular extension, which has the function of depressing the tongue, so that the treatment can be carried out without interference. A further embodiment of the applicator (apparatus) of the present invention has a cylindrical configuration and can thus serve for the effective treatment of the mucous membranes in the rectal, anal and vaginal regions.

The following description of exemplary embodiments is not to be construed as limiting, but illustrates the use of the apparatus.

The apparatus according to the present invention can be used in all regions of the body and locations on the body in which bacterial inflammation can form, i.e. preferably such regions in which mucous membranes are present. Sutures, which serve for joining together previously severed tissue portions, can be created by using string-like devices. The sutures of this type dispense the photosensitizer which then adheres to any bacteria present. One end of these sutures is then connected to a suitable light source. The thus transported light is emitted along the suture and the dyed bacteria and microorganisms are successfully destroyed.

Catheters, at least all catheters insertable into the body, such as urinal catheters and endoscopes, cannulas, stents, other implants, i.e. foreign bodies, which are inserted into the body and can deposit germs or provoke inflammation there, can now be manufactured in such a way that the product corresponds to the apparatus according to the present invention. The manufactured apparatuses can then be connected to a suitable light source and potential inflammation can be successfully treated.

The apparatus according to the present invention can thus assume a variety of forms, as described above, which can all be closely adapted to the body to be treated.

Some embodiments, such as a solid-core, cylindrical hollow body, tube-like and plate-like, foil-like applicator, should be mentioned in an exemplary manner as a summary.

The distribution of the photosensitizer can be carried out by means of flushing and/or suction, by means of diffusion with the impregnated applicator, and by means of inhalation and/or suction.

The apparatus according to the present invention or the applicators, are used with skin/mucous membrane surfaces (burns, wounds, accessible mucous membranes, such as the upper respiratory organs, such as pharynx, gut, vagina, draining urethral tract, other endoscopically accessible body regions, that can also include body regions, which are accessible, for example, by means of a micro robot. Furthermore, the upper and further regions of the gastro-intestinal tract, such as the oesophagus, stomach, duodenum etc., are to be mentioned as regions of use.

Externally, the apparatus according to the present invention is to be used on the skin. The inner hollow organs such as the stomach, intestine etc. also count among the fields of use. Blood vessels become fields of use of the apparatus according to the present invention by means of correspondingly adapted cannulas or other types of applicators.

### DESCRIPTION OF THE DRAWING

Exemplary embodiments of the present invention will be discussed in the following in a non-limiting manner with reference to the accompanying drawing, in which:

FIG. 1 schematically shows a y-shaped applicator.

### DESCRIPTION OF AN EMBODIMENT

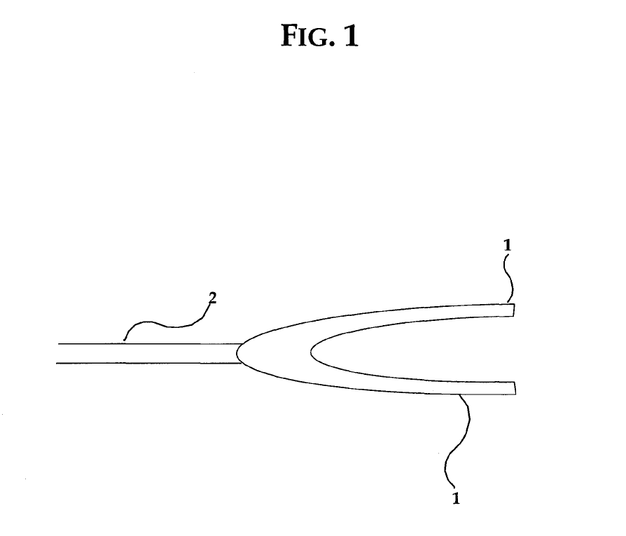

FIG. 1 schematically shows a y-shaped applicator. It is useful for the application of photodynamic therapy in the nasal cavity. For this purpose, one end of the Y-prong 1 is inserted into the right nostril and the other end of the Y-prong is inserted into the left nostril. The supply line 2 for the energy and flushing fluid supply is schematically shown in the figure. After dying the nasal mucous membrane with the photosensitizer, it can be flushed with a flushing fluid in order to flush out the excess dye. Both ends of the Y-prong of the applicator are equipped with a flushing device, a suction device and the light source. In order to avoid the flushing fluid carrying the bacteria to other regions of the body, only one end of the applicator is used for flushing and the other end is used for directly sucking the flushing fluid back in. By these means the flushing fluid contaminated with bacteria cannot be further spatially distributed and form new sites of infection. The flow of flushing fluid is thus directed and guided.

1-19. (canceled)

20. An apparatus for carrying out antibacterial photodynamic therapy, wherein the apparatus comprises:
an illumination means; and
a means for dispensing the photosensitizer,
wherein
the illumination means is an optical fiber emitting the light at the desired frequency through its side walls,
the means for dispensing the photosensitizer is adapted such that the photosensitizer is dispensed in spatial proximity to the body region to be treated,
the one flushing device for removing the excess dye, i.e. the photosensitizer, comprises a suction device, which sucks most of the flushing fluid and the excess dye dissolved therein back in,
the means for dispensing the photosensitizer, preferably a tube, sheaths the light emitting portion of the illumination means, and
the sheathing material at least partially transmits the light emitted by the illumination means.

21. The apparatus according to claim 20, wherein the means for dispensing the photosensitizer comprises at least one tube, which conducts the photosensitizer from a source via the tube to the body regions to be treated, wherein the tube can comprise at least one outlet for dispensing the photosensitizer, but can also comprise an indefinite maximum number of outlets for dispensing the photosensitizer.

22. The apparatus according to claim 20, wherein the means for dispensing the photosensitizer, preferably a tube, sheaths the light emitting portion of the illumination means, wherein the sheathing material at least partially transmits the light emitted by the illumination means.

23. The apparatus according to claim 20, wherein the illumination means is coated with the photosensitizer.

24. The apparatus according to claim 20, wherein the illumination means is sheathed with a material, wherein the sheathing material consists of a fabric, which has absorbed the photosensitizer and, in turn, dispenses the latter to the body regions to be treated.

25. The apparatus (applicator) for carrying out antibacterial photodynamic therapy according to claim 20, wherein the apparatus (the applicator) is adapted to the shape of each body cavity to be treated.

26. The apparatus (applicator) for carrying out antibacterial photodynamic therapy according to claim 20, wherein the flushing device comprises an ultrasonic nebulizer.

27. The apparatus (applicator) for carrying out antibacterial photodynamic therapy in the nasal cavity according to claim 20, characterized in that the apparatus (the applicator) has a y-shaped configuration and each of the two upper ends of the y-shaped apparatus (of the y-shaped applicator) has its own flushing and/or suction device and a light source.

28. The apparatus (applicator) according to claim 20, wherein the flushing and/or suction device of one end of the apparatus (of one end of the applicator) (1) flushes whenever the flushing and/or suction device of the other end of the apparatus (of the end of the applicator) (1) sucks in the flushing fluid.

29. The apparatus (applicator) according to claim 20, wherein the flushing device is configured as an inhalation apparatus.

30. The apparatus (applicator) according to claim 20, wherein the flushing and/or suction device of one end of the apparatus (one end of the applicator) flushes whenever the flushing and/or suction device of the other end of the apparatus (other end of the applicator) sucks in the flushing fluid.

31. The apparatus (applicator) for carrying out antibacterial photodynamic therapy in the oral and pharyngeal cavities according to claim 20, wherein the apparatus (applicator) has the same form as the dental ridge.

32. The apparatus (applicator) according to claim 20, wherein the apparatus comprises a cylindrical configuration.
